(19) United States
(12) Patent Application Publication
(10) Pub. No.: US 2018/0057756 A1
(43) Pub. Date: Mar. 1, 2018

(54) A POLAR ADDITIVE-CONTAINING HYDROPROCESSING CATALYST AND METHOD OF MAKING AND USING THEREOF

(71) Applicant: SHELL OIL COMPANY, HOUSTON, TX (US)

(72) Inventors: Karl Marvin KRUEGER, Houston, TX (US); Thomas WEBER, Amsterdam (NL)

(21) Appl. No.: 15/681,563

(22) Filed: Aug. 21, 2017

Related U.S. Application Data
(60) Provisional application No. 62/378,311, filed on Aug. 23, 2016.

(51) Int. Cl. C10G 45/08; C07D 319/06; C07D 207/267

(52) U.S. Cl. CPC C10G 45/08; C07D 207/267; C07D 319/06

(57) ABSTRACT

A method of making a hydroprocessing catalyst composition that includes impregnating a support material with metals and an organic acid compound followed by a controlled drying of the impregnated support and incorporation thereafter of a polar additive into the dried, impregnated support. The hydroprocessing catalyst is made by this method.

The present non-provisional application claims priority from U.S. Patent Application No. 62/378,311, filed Aug. 23, 2016, incorporated herein by reference.

This invention relates to a hydroprocessing catalyst composition that is impregnated with a polar additive, a method of making and use of the polar additive-containing catalyst.

There has been great effort by industry to find new hydrotreating catalyst compositions and formulations that may be used in the hydroprocessing of distillate and gas oil feedstocks to reduce sulfur concentrations, and, in particular, to provide low-sulfur diesel and other products.

An especially good catalyst composition that is useful in deep hydrodesulfurization and in other methods of hydrotreating hydrocarbon feedstocks is disclosed in U.S. Pat. No. 8,262,905. The '905 patent discloses a catalyst composition that includes a support material comprising a metal component, hydrocarbon oil, and a polar additive. This catalyst composition is made by incorporating a metals impregnation solution into a support material followed by drying and incorporating the polar additive into the dried impregnated support material.

Another very good catalyst composition that has application in the hydroprocessing of hydrocarbon feedstocks is disclosed in US 2014/0353213. This catalyst composition comprises a support material that is either loaded with an active metal precursor or contains a metal component of a metal salt solution and a heterocyclic additive. The catalyst composition is made by incorporating a metals impregnation solution into a support material followed by incorporating an additive that is a heterocyclic compound. The metals-incorporated support material may be dried prior to incorporating therein the heterocyclic compound.

There is a continued need to find improved higher activity hydrotreating catalysts. There is also need to find improved methods of manufacturing these higher activity hydrotreating catalysts.

Accordingly, provided is a hydroprocessing catalyst composition that comprises a dried, impregnated support material and a polar additive having a dipole moment of at least 0.45 that is incorporated into the dried, impregnated support material. The impregnated support material, comprises a support material, a Group 9/10 metal component, a Group 6 metal component, and an organic acid compound, and has been dried at a drying temperature in the range of from above the melting temperature of the organic acid compound and below the boiling temperature of the organic acid compound to provide the dried, impregnated support material of the hydroprocessing catalyst composition prior to incorporation therein of the polar additive.

The hydroprocessing catalyst composition is made by a method comprising the step of incorporating an impregnation solution, comprising a Group 9/10 metal component, a Group 6 metal component, and an organic acid compound, into a support material to provide an impregnated support material. The impregnated support material is then dried at a drying temperature that is in the range of from above the melting temperature of the organic acid compound and below the boiling temperature of the organic acid compound to provide a dried impregnated support material followed by incorporating a polar additive having a dipole moment of at least 0.45 into the dried impregnated support material to thereby provide an additive-impregnated composition.

The hydroprocessing catalyst composition has application and use in processes for treating hydrocarbon feedstocks that include a step of contacting the inventive catalyst or a catalyst made by the inventive method with the hydrocarbon feedstock under suitable hydroprocessing process conditions.

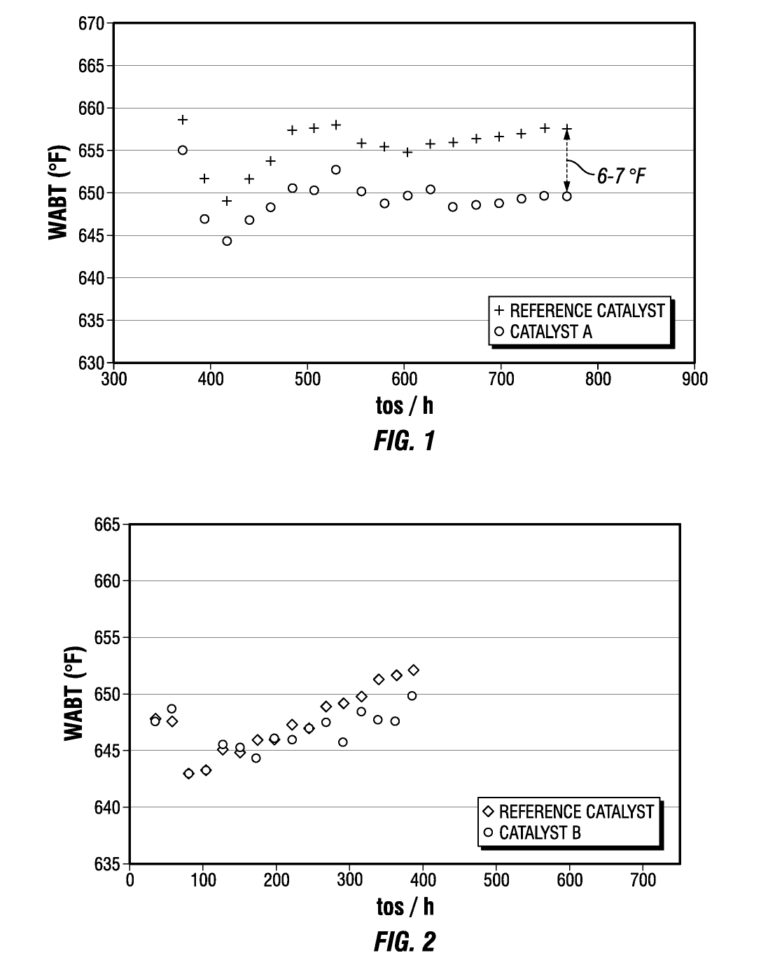

FIG. 1 presents comparison plots of the adjusted weighted average bed temperature (WABT) over time for the hydrodesulfurization of a gas oil feedstock to yield a product having a reduced sulfur concentration of 10 ppmw with one plot representing Catalyst A (citric acid) and the other plot representing the comparison Reference Catalyst.

FIG. 2 presents comparison plots of the adjusted weighted average bed temperature (WABT) over time for the hydrodesulfurization of a gas oil feedstock to yield a product having a reduced sulfur concentration of 10 ppmw with a plot representing Catalysts B (gluconic acid) and the other plot representing the comparison Reference Catalysts.

The additive-impregnated composition of the invention is prepared by incorporating a metals impregnation solution that contains an organic acid (as described in detail elsewhere herein) into a support material followed by conducting a controlled drying of the resulting impregnated support material so as to provide a dried, impregnated support material, which is then impregnated with a polar additive so as to provide the additive-impregnated composition.

It has been discovered that the catalytic activity of already highly active polar additive and heterocyclic additive-containing hydroprocessing catalysts is significantly enhanced by the addition of certain molten organic acids to the metals impregnation solution used to incorporate catalytic metals into the support material of the additive-containing or additive-impregnated catalyst composition of the invention.

In preparing the additive-impregnated composition of the invention, the application of the organic acid, by its inclusion with or addition to the metals impregnation solution used to incorporate metals into the support material of the composition, further requires the critical control of the drying temperature at which the impregnated support material is dried prior to incorporating the polar additive into the resulting dried impregnated support material. The drying temperature is controlled so that it is above the melting temperature of the organic acid of the metals impregnation solution, but the drying temperature is also maintained below the boiling temperature of the organic acid.

It is believed that the combination of using the organic acid in the metals impregnation solution and carefully controlling the drying temperature at which the impregnated support is dried so that it exceeds the melting temperature of the organic acid contained in the impregnation solution, but only by a small temperature differential, provides a final, additive-impregnated composition having enhanced desulfurization activity compared to prior art additive-impregnated compositions. The temperature differential should be such that the upper end of the range for the drying temperature is less than the boiling temperature of the organic acid.

The dried, impregnated support material preferably comprises a support material, a Group 9/10 metal component, a Group 6 metal component and an organic acid compound. As noted above, the use of the organic acid compound in the preparation of the dried, impregnated support material provides for a final additive-impregnated composition that has enhanced desulfurization activity compared to other additive-impregnated compositions that are prepared without the use of an organic acid compound in the metals-impregnation solution.

The support material of the inventive composition can comprise any suitable inorganic oxide material that is typically used to carry catalytically active metal components. Examples of possible useful inorganic oxide materials include alumina, silica, silica-alumina, magnesia, zirconia, boria, titania and mixtures of any two or more of such inorganic oxides. The preferred inorganic oxides for use in the formation of the support material are alumina, silica, silica-alumina and mixtures thereof. Most preferred, however, is alumina.

In the preparation of the inventive composition, the metal components are incorporated into the support material by any suitable method or means for loading or incorporating the metal components into the support material. In a preferred method, the support material is impregnated with the metal components using any of the known impregnation methods, such as, by the incipient wetness method, to incorporate the metals into the support material. It is a necessary feature of the invention to incorporate the organic acid compound into the support material along with the metal compound prior to drying the impregnated support material under controlled drying conditions to provide the dried, impregnated support material into which a polar additive is incorporated.

It is preferred for the support material into which the metal and organic acid components are incorporated to first be formed into a shaped particle comprising the inorganic oxide material followed by loading the shaped particle with the metal and organic acid components. This is preferably done by impregnation of the shaped particle with an aqueous solution of metal salts of Group 9/10 metal and Group 6 metal and of the organic acid to give the support material that comprises a metal of a metal salt solution and an organic acid.

To form the shaped particle, the inorganic oxide material, which preferably is in powder form, is mixed with water and, if desired or needed, a peptizing agent and/or a binder to form a mixture that can be shaped into an agglomerate. It is desirable for the mixture to be in the form of an extrudable paste suitable for extrusion into extrudate particles, which may be of various shapes such as cylinders, trilobes, etc. and nominal sizes such as 1/16″, ⅛″, 3/16″, etc. The support material of the inventive composition, thus, preferably, is a shaped particle comprising an inorganic oxide material.

The shaped particle is then dried under standard drying conditions that can include a drying temperature in the range of from 50° C. to 200° C., preferably, from 75° C. to 175° C., and, most preferably, from 90° C. to 150° C.

After drying, the shaped particle is calcined under standard calcination conditions that can include a calcination temperature in the range of from 250° C. to 900° C., preferably, from 300° C. to 800° C., and, most preferably, from 350° C. to 600° C.

The calcined shaped particle can have a surface area (determined by the BET method employing N₂, ASTM test method D 3037) that is in the range of from 50 m²/g to 450 m²/g, preferably from 75 m²/g to 400 m²/g, and, most preferably, from 100 m²/g to 350 m²/g.

The mean pore diameter in angstroms (Å) of the calcined shaped particle is in the range of from 50 to 200, preferably, from 70 to 150, and, most preferably, from 75 to 125.

The pore volume of the calcined shaped particle is in the range of from 0.5 cc/g to 1.1 cc/g, preferably, from 0.6 cc/g to 1.0 cc/g, and, most preferably, from 0.7 to 0.9 cc/g.

Less than ten percent (10%) of the total pore volume of the calcined shaped particle is contained in the pores having a pore diameter greater than 350 Å, preferably, less than 7.5% of the total pore volume of the calcined shaped particle is contained in the pores having a pore diameter greater than 350 Å, and, most preferably, less than 5%.

The references herein to the pore size distribution and pore volume of the calcined shaped particle are to those properties as determined by mercury intrusion porosimetry, ASTM test method D 4284. The measurement of the pore size distribution of the calcined shaped particle is by any suitable measurement instrument using a contact angle of 140° with a mercury surface tension of 474 dyne/cm at 25° C.

The calcined shaped particle is impregnated with the metal components of the composition in one or more impregnation steps and using one or more aqueous solutions containing at least one metal salt wherein the metal compound of the metal salt solution is an active metal or active metal precursor. In a further embodiment, any one or more of the aqueous solutions contain an organic acid compound or, alternatively, the organic acid compound is impregnated into the calcined shaped particle using a separately prepared aqueous solution containing the organic acid compound.

The metal elements are those selected from Group 6 of the IUPAC Periodic Table of the elements (e.g., chromium (Cr), molybdenum (Mo), and tungsten (W)) and Groups 9 and 10 (Group 9/10) of the IUPAC Periodic Table of the Elements (e.g., cobalt (Co) and nickel (Ni)). Phosphorous (P) is also a desired metal component.

For the Group 9/10 metals, the metal salts include Group 9 or 10 metal acetates, formats, citrates, oxides, hydroxides, carbonates, nitrates, sulfates, and two or more thereof. The preferred metal salts are metal nitrates, for example, such as nitrates of nickel or cobalt, or both.

For the Group 6 metals, the metal salts include Group 6 metal oxides or sulfides. Preferred are salts containing the Group 6 metal and ammonium ion, such as ammonium heptamolybdate and ammonium dimolybdate.

The organic acid compound incorporated into the support material is any suitable organic acid that is soluble in water and provides in combination with the other features of the invention the benefits discussed herein. Preferably, the organic acid compound is selected from the group of carboxylic compounds consisting of di or tri carboxylic compounds having at least 3 up to 10 carbon atoms. The carboxylic compounds further may or may not be substituted with one or more hydroxyl groups.

A further desirable property of the organic acid is for it to have a reasonably low but not too low melting temperature. Generally, the melting temperature of the organic acid ought to be in the range of from 100° C. to 200° C., preferably, from 110° C. to 190° C., and, more preferably, from 120° C. to 180° C.

The boiling temperature of the organic acid may be as high as 350° C. or higher. Typically, the boiling temperature is within the range of from 210° C. to 340° C., or from 230° C. to 330° C.

The organic acid compound further should have a molar mass in the range of from 100 g/mol to 250 g/mol. More specifically, the molar mass of the organic acid compound is in the range of from 120 g/mol to 240 g/mol, and, most specifically from 140 g/mol to 230 g/mol.

Examples of preferred organic acid compounds include citric acid and gluconic acid.

The concentration of the metal compounds in the impregnation solution is selected to provide the desired metal content in the final composition of the invention. In preparing the impregnation solution, consideration is given to the pore volume of the support material into which the aqueous solution is impregnated and to the amount of organic acid required to provide the benefits discussed herein. Typically, the concentration of metal compound in the impregnation solution is in the range of from 0.01 to 100 moles per liter. The concentration of the organic acid compound in the impregnation solution is that required to provide the desired concentration within the final composition of the invention.

The metal content of the support material having a metal component incorporated therein may depend upon the application for which the final additive-impregnated composition of the invention is used. Generally, however, for hydroprocessing applications, the Group 9/10 metal component, i.e., cobalt or nickel, or both, can be present in the support material having a metal component incorporated therein in an amount in the range of from 0.5 wt. % to 20 wt. %, preferably from 1 wt. % to 15 wt. %, and, most preferably, from 2 wt. % to 12 wt. %.

The Group 6 metal component, i.e., molybdenum or tungsten, preferably, molybdenum, can be present in the support material having a metal component incorporated therein in an amount in the range of from 5 wt. % to 50 wt. %, preferably from 8 wt. % to 40 wt. %, and, most preferably, from 12 wt. % to 30 wt. %.

The values for weight percent referenced above for the metal components are based on the total dry weight of the additive-impregnated composition and the metal component as the element regardless of the actual form of the metal component.

Before incorporating the polar additive into the support material, it is an important aspect of the invention to dry the impregnated support that comprises the support material having been impregnated with the metal and organic acid components. It is an important feature of the method of preparing the additive-impregnated composition to dry the impregnated support material under controlled drying conditions such that the drying temperature is above the melting temperature of the organic acid component but less than the boiling temperature of the organic acid component.

This drying step prepares the impregnated support material for incorporating the polar additive into the support material. It is important that the organic acid compound is unconverted and remains on the support material when the polar additive is placed onto the support material. Thus, as noted above, the drying temperature should be maintained only slightly above the melting temperature of the organic acid compound, which typically is a small temperature differential above the melting temperature, and below the boiling temperature of the organic acid compound. This temperature differential is generally less than 50° C., preferably, the temperature differential is less than 30° C. and, more preferably, it is less than 20° C.

The polar additive used in the preparation of the inventive composition can be any suitable molecule that provides for the benefits and has the characteristic molecular polarity or molecular dipole moment and other properties, if applicable, as are described herein. Examples of suitable polar additive compounds that may be used in the preparation of the composition of the invention are disclosed and described in U.S. Pat. No. 8,262,905, issued 11 Sep. 2012, and in US Pub. US 2014/0353213, published 4 Dec. 2014, both of which are incorporated herein by reference.

Molecular polarity is understood in the art to be a result of non-uniform distribution of positive and negative charges of the atoms that make up a molecule. The dipole moment of a molecule may be approximated as the vector sum of the individual bond dipole moments, and it can be a calculated value.

One method of obtaining a calculated value for the dipole moment of a molecule, in general, includes determining by calculation the total electron density of the lowest energy conformation of the molecule by applying and using gradient corrected density functional theory. From the total electron density, the corresponding electrostatic potential is derived and point charges are fitted to the corresponding nuclei. With the atomic positions and electrostatic point charges known, the molecular dipole moment can be calculated from a summation of the individual atomic moments.

As the term is used in this description and in the claims, the “dipole moment” of a given molecule is that as determined by calculation using the publicly available, under license, computer software program named Materials Studio, Revision 4.3.1, copyright 2008, Accerlys Software Inc.

Following below is a brief discussion of some of the technical principles behind the computation method and application of the Materials Studio computer software program for calculating molecular dipole moments.

The first step in the determination of the calculated value of the dipole moment of a molecule using the Materials Studio software involves constructing a molecular representation of the compound using the sketching tools within the visualizer module of Materials Studio. This sketching process involves adding atoms to the sketcher window that constitute the compound and completing the bonds between these atoms to fulfill the recognized bonding connectivity that constitute the compound. Using the clean icon within the Material Studio program automatically orients the constructed compound into the correct orientation. For complex compounds, a conformational search is performed to ensure that the orientation used to calculate the molecular dipole is the lowest energy conformation, i.e., its natural state.

The quantum mechanical code DMol3 (Delley, B. J. Chem. Phys., 92, 508 (1990)) is utilized to calculate the molecular dipole moments from first principles by applying density functional theory. Density functional theory begins with a theorem by Hohenberg and Kohn (Hohenberg, P. et al., “Inhomogeneous Electron Gas”, Phys. Rev. B, 136, 864-871 (1964); Levy, M., “Universal Variational Functionals of Electron Densities, First-Order Density Matrices, and Natural Spin-Orbitals and Solution of the V-Representability Problem”, Proc. Natl. Acad. Sci. U.S.A., 76, 6062-6065 (1979)), which states that all ground-state properties are functions of the charge density P. Specifically, the total energy Eₜ may be written as:

Eₜ[ρ]=T[ρ]+U[ρ]+E_{xc}[ρ] Eq.1

where T[ρ] is the kinetic energy of a system of noninteracting particles of density ρ, U[ρ] is the classical electrostatic energy due to Coulombic interactions, and E_{xc}[ρ] includes all many-body contributions to the total energy, in particular the exchange and correlation energies. As in other molecular orbital methods (Roothaan, C. C. J., “New Developments in Molecular Orbital Theory”, Rev. Mod. Phys., 23, 69-89 (1951); Slater, J. C., “Statistical Exchange-Correlation in the Self-Consistent Field”, Adv. Quantum Chem., 6, 1-92 (1972); Dewar, M. J. S. J. Mol. Struct., 100, 41 (1983)), the wave function is taken to be an antisymmetrized product (Slater determinant) of one-particle functions, that is, molecular orbitals:

Ψ=A(n)|φ₁(1)φ₂(2) . . . φₙ(n) Eq.2

The molecular orbitals also must be orthonormal:

[symbol]

φᵢ|φⱼ [symbol] =δᵢⱼ Eq.3

The charge density summed over all molecular orbitals is given by the simple sum:

$$ρ ( r ) = {\sum }_{i} {\vert {φ}_{i} ( r ) \vert}^{2} \qquad Eq . 4$$

where the sum goes over all occupied molecular orbitals φᵢ. The density obtained from this expression is also known as the charge density. From the wave functions and the charge density the energy components from Eq. 1 can be written (in atomic units) as:

$$T = 〈 {\sum }_{i}^{n} {φ}_{i} \vert \frac{- {∇}^{2}}{2} \vert {φ}_{i} 〉 \qquad Eq . 5$$

In Eq. 6, Zα refers to the charge on nucleus α of an N-atom system. Further, in Eq. 6, the term ρ(r₁)V_{N}, represents the electron-nucleus attraction, the term ρ(r₁)Vₑ(r₁)/2, represents the electron-electron repulsion, and the term, V_{NN}, represents the nucleus-nucleus repulsion.

$$U = {\sum }_{i}^{n} {\sum }_{α}^{N} 〈 {φ}_{i} ( r ) \vert \frac{- Z}{{R}_{α} - r} \vert {φ}_{i} ( r ) 〉 + \frac{1}{2} {\sum }_{i} {\sum }_{j} 〈 {φ}_{i} ( {r}_{1} ) {φ}_{j} ( {r}_{2} ) \frac{1}{{r}_{1} - {r}_{2}} {φ}_{i} ( {r}_{1} ) {φ}_{j} ( {r}_{2} ) 〉 + {\sum }_{α}^{N} {\sum }_{β < α} \frac{{Z}_{α} {Z}_{β}}{\vert {R}_{α} - R} = - {\sum }_{α}^{N} 〈 ρ ( {r}_{1} ) \frac{{Z}_{α}}{\vert {R}_{α} - {r}_{1} \vert} 〉 + \frac{1}{2} 〈 ρ ( {r}_{1} ) ρ ( {r}_{2} ) \frac{1}{\vert {r}_{1} - {r}_{2} \vert} 〉 + {\sum }_{α}^{N} {\sum }_{β < α} \frac{{Z}_{α} \text{?}}{\vert {R}_{α} - \text{?}} ≡ 〈 - ρ ( {r}_{1} ) {V}_{N} 〉 + 〈 ρ ( {r}_{1} ) \frac{{V}_{e} ( {r}_{1} )}{2} 〉 + {V}_{NN} \\ \text{?} \text{indicates text missing or illegible when filed} \qquad Eq . 6$$

The term, E_{xc}[ρ] in Eq. 1, the exchange-correlation energy, requires some approximation for this method to be computationally tractable. A simple and surprisingly good approximation is the local density approximation, which is based on the known exchange-correlation energy of the uniform electron gas. (Hedin, L. et al., “Explicit Local Exchange Correlation Potentials”, J. Phys. C, 4, 2064-2083 (1971); Ceperley, D. M. et al, “Ground State of the Electron Gas By a Stochastic Method”, Phys. Rev. Lett., 45, 566-569 (1980)). The local density approximation assumes that the charge density varies slowly on an atomic scale (i.e., each region of a molecule actually looks like a uniform electron gas). The total exchange-correlation energy can be obtained by integrating the uniform electron gas result:

ε_{xc}[ρ]≅∫ρ(r)ε_{xc}[ρ(r)]dr Eq.7

where E_{xc}[ρ] is the exchange-correlation energy per particle in a uniform electron gas and ρ is the number of particles. In this work the gradient corrected exchange-correlation functional PW91 is used (Perdew, J. P. & Wang, Y., Phys. Rev. B, 45, 13244 (1992)).

With all the components derived to describe the total energy of any molecular system within the density functional formalism, the dipole moment is calculated from a summation of the individual electronic and nuclear dipole moment vectors that are displayed at the end of the DMol3 output file.

References herein to the polar additive are understood to mean a molecule that has polarity and having a dipole moment, as calculated by the aforementioned Materials Studio software or other known method that will provide substantially the same calculated value for the dipole moment of a molecule as the Materials Studio software will provide, which exceeds the dipole moment of the hydrocarbon oil that is used in the inventive composition.

The dipole moment of the polar additive should be at least or exceed 0.45. However, it is preferred for the polar additive to have a characteristic dipole moment that is at least or exceeds 0.5, and, more preferred, the dipole moment of the polar additive should be at least or exceed 0.6. A typical upper limit to the dipole moment of the polar additive is up to about 5, and, thus, the dipole moment of the polar additive may be, for example, in the range of from 0.45 to 5. It is preferred for the dipole moment of the polar additive to be in the range of from 0.5 to 4.5, and, more preferred, the dipole moment is to be in the range of from 0.6 to 4.

A particularly desirable attribute of the polar additive is for it to be a heterocompound. A heterocompound is considered herein to be a molecule that includes atoms in addition to carbon and hydrogen. These additional atoms can include, for example, nitrogen or oxygen, or both. It is desirable for the group of heterocompounds to exclude those heterocompounds that include sulfur, and, in all cases, the polar additive does not include paraffin and olefin compounds, i.e. compounds that contain only carbon and hydrogen atoms. Considering the exclusion of sulfur-containing compounds from the definition of the group of heterocompounds, it can further be desirable for the oil and additive impregnated composition, before its treatment with hydrogen and sulfur, to exclude the material presence of a sulfur-containing compound.

Another preferred characteristic of the polar additive is for its boiling temperature to be in the range of from 50° C. to 275° C. Preferably, the boiling temperature of the polar additive is to be in the range of from 60° C. to 250° C., and, more preferably, from it is in the range of from 80° C. to 225° C.

Certain heterocyclic compounds are particularly suitable for use as the polar additive of the invention. Such suitable heterocyclic, polar compounds include those that provide for the benefits and have the characteristic properties as described herein. Specifically, the heterocyclic compound additive of the composition is selected from the group of heterocyclic, polar compounds having the formula: CₓHₙN_{y}O_{z}, wherein: x is an integer of 3 or larger; y is either zero or an integer in the range of from 1 to 3 (i.e., 0, 1, 2, or 3); z is either zero or an integer in the range of from 1 to 3 (i.e., 0, 1, 2, or 3); and n is the number of hydrogen atoms required to fill the remaining bonds with the carbon atoms of the molecule.

Preferred polar additive compounds are heterocyclic compounds containing either nitrogen or oxygen as the heteroatom member of its ring, such as molecular compounds having either a lactam structure or a cyclic ester structure or a cyclic ether structure. The lactam compounds, or cyclic amides, may include compounds having such general structures as β-lactam, γ-lactam, and δ-lactam in which the nitrogen atom may instead of a hydrogen atom have bonded thereto an alkyl group having from 1 to 6 or more carbon atoms and any of the carbon atoms, other than the carbonyl moiety, present in the ring structure may have bonded thereto an alkyl group having from 1 to 6 or more carbon atoms.

The cyclic ether compounds, or oxacycloalkanes, may include cyclic compounds in which one or more of the carbon atoms within the ring structure is replaced with an oxygen atom. The cyclic ether compound may also include within the ring a carbonyl moiety or any one or more of the carbon atoms present in the ring structure may have bonded thereto an alkyl group having from 1 to 6 or more carbon atoms, or the ring may include both a carbonyl moiety and one or more carbon atoms having bonded thereto an alkyl group having from 1 to 6 or more carbon atoms.

The cyclic ester compounds may include lactone compounds that fit the structure presented above, for example, β-propiolactone, γ-butyrolactone, and δ-valerolactone. The cyclic ester compounds further may include the cyclic esters having more than one oxygen atom contained within the ring structure.

More preferred polar additive compounds are those heterocyclic compounds in which the heteroatom is either oxygen or nitrogen.

Examples of more preferred compounds include propylene carbonate, e.g., a cyclic ester compound, and N-methylpyrrolidone, e.g. a cyclic amide compound.

To provide the additive-impregnated composition of the invention, the polar additive is incorporated into the dried, impregnated support material. The polar additive is used to fill a significant portion of the available pore volume of the pores of the dried, impregnated support material that is loaded with the metals and organic acid, to thereby provide a composition that comprises, or consists essentially of, or consists of, a support material, a Group 9/10 metal component, a Group 6 metal component, and an organic acid compound.

The preferred method of impregnating the dried, impregnated support material with the polar additive may be any standard well-known pore fill methodology whereby the pore volume is filled by taking advantage of capillary action to draw the liquid into the pores of the metal loaded support material. It is desirable to fill at least 75% of the available pore volume of the metal loaded support material with the polar additive. It is preferred for at least 80% of the available pore volume of the dried, impregnated support material to be filled with the polar additive, and, most preferred, at least 90% of the pore volume is filled with the polar additive.

The additive-impregnated composition may be installed, as is, into a reactor vessel or within a reactor system that is to undergo a start-up procedure in preparation of or prior to the introduction of a sulfiding feed that can include a sulfiding agent or a hydrocarbon feedstock containing a concentration of an organic sulfur compound.

The additive-impregnated composition of the invention may be treated, either ex situ or in situ, with hydrogen and with a sulfur compound. The additive-impregnated composition can be activated, in situ, by a hydrogen treatment step followed by a sulfurization step. As earlier noted, the additive-impregnated composition can first undergo a hydrogen treatment that is then followed with treatment with a sulfur compound.

The hydrogen treatment includes exposing the additive-impregnated composition to a gaseous atmosphere containing hydrogen at a temperature ranging upwardly to 250° C. Preferably, the additive-impregnated composition is exposed to the hydrogen gas at a hydrogen treatment temperature in the range of from 100° C. to 225° C., and, most preferably, the hydrogen treatment temperature is in the range of from 125° C. to 200° C.

The partial pressure of the hydrogen of the gaseous atmosphere used in the hydrogen treatment step generally can be in the range of from 1 bar to 70 bar, preferably, from 1.5 bar to 55 bar, and, most preferably, from 2 bar to 35 bar. The additive-impregnated composition is contacted with the gaseous atmosphere at the aforementioned temperature and pressure conditions for a hydrogen treatment time period in the range of from 0.1 hours to 100 hours, and, preferably, the hydrogen treatment time period is from 1 hour to 50 hours, and most preferably, from 2 hours to 30 hours.

Sulfiding of the additive-impregnated composition after it has been treated with hydrogen can be done using any conventional method known to those skilled in the art. Thus, the hydrogen treated additive-impregnated composition can be contacted with a sulfur-containing compound, which can be hydrogen sulfide or a compound that is decomposable into hydrogen sulfide, under the contacting conditions of the invention. Examples of such decomposable compounds include mercaptans, CS₂, thiophenes, dimethyl sulfide (DMS), and dimethyl disulfide (DMDS).

Also, preferably, the sulfiding is accomplished by contacting the hydrogen treated composition, under suitable sulfurization treatment conditions, with a hydrocarbon feedstock that contains a concentration of a sulfur compound. The sulfur compound of the hydrocarbon feedstock can be an organic sulfur compound, particularly, one which is typically contained in petroleum distillates that are processed by hydrodesulfurization methods.

Suitable sulfurization treatment conditions are those which provide for the conversion of the active metal components of the hydrogen treated additive-impregnated composition to their sulfided form. Typically, the sulfiding temperature at which the hydrogen treated additive-impregnated composition is contacted with the sulfur compound is in the range of from 150° C. to 450° C., preferably, from 175° C. to 425° C., and, most preferably, from 200° C. to 400° C.

The additive-impregnated composition of the invention, after its treatment with hydrogen and sulfur, is a highly effective catalyst for use in the hydrotreating of hydrocarbon feedstocks. This catalyst is particularly useful in applications involving the hydrodesulfurization and hydrodenitrogenation of hydrocarbon feedstocks, and, especially, it has been found to be an excellent catalyst for use in the hydrodesulfurization of distillate feedstocks, in particular, diesel, to make an ultra-low sulfur distillate product having a sulfur concentration of less than 15 ppmw, preferably, less than 10 ppmw, and, most preferably, less than 8 ppmw.

In the hydrotreating applications, the additive-impregnated composition that is treated with hydrogen and sulfur, as described above, is contacted under suitable hydrodesulfurization or hydrodenitrogenation, or both, conditions with a hydrocarbon feedstock that typically has a concentration of sulfur or nitrogen, or both.

The more typical and preferred hydrocarbon feedstock processed with the additive-impregnated composition is a petroleum middle distillate cut having a boiling temperature at atmospheric pressure in the range of from 140° C. to 410° C. These temperatures are approximate initial and boiling temperatures of the middle distillate. Examples of refinery streams intended to be included within the meaning of middle distillate include straight run distillate fuels boiling in the referenced boiling range, such as, kerosene, jet fuel, light diesel oil, heating oil, heavy diesel oil, and the cracked distillates, such as FCC cycle oil, coker gas oil, and hydrocracker distillates. The preferred feedstock of the inventive distillate hydrotreating process is a middle distillate boiling in the diesel boiling range of from about 140° C. to 400° C.

The sulfur concentration of the middle distillate feedstock can be a high concentration, for instance, being in the range upwardly to about 2 weight percent of the distillate feedstock based on the weight of elemental sulfur and the total weight of the distillate feedstock inclusive of the sulfur compounds. Typically, however, the distillate feedstock of the inventive process has a sulfur concentration in the range of from 0.01 wt. % (100 ppmw) to 1.8 wt. % (18,000). But, more typically, the sulfur concentration is in the range of from 0.1 wt. % (1000 ppmw) to 1.6 wt. % (16,000 ppmw), and, most typically, from 0.18 wt. % (1800 ppmw) to 1.1 wt. % (11,000 ppmw).

It is understood that the references herein to the sulfur content of the distillate feedstock are to those compounds that are normally found in a distillate feedstock or in the hydrodesulfurized distillate product and are chemical compounds that contain a sulfur atom and which generally include organosulfur compounds.

Also, when referring herein to “sulfur content” or “total sulfur” or other similar reference to the amount of sulfur that is contained in a feedstock, product or other hydrocarbon stream, what is meant is the value for total sulfur as determined by the test method ASTM D2622-10, entitled “Standard Test Method for Sulfur in Petroleum Products by Wavelength Dispersive X-ray Fluorescence Spectrometry.” The use of weight percent (wt. %) values of this specification when referring to sulfur content correspond to mass % values as would be reported under the ASTM D2622-10 test method.

The middle distillate feedstock may also have a concentration of nitrogen compounds. When it does have a concentration of nitrogen compounds, the nitrogen concentration may be in the range of from 15 parts per million by weight (ppmw) to 3500 ppmw. More typically for the middle distillate feedstocks that are expected to be handled by the process, the nitrogen concentration of the middle distillate feedstock is in the range of from 20 ppmw to 1500 ppmw, and, most typically, from 50 ppmw to 1000 ppmw.

When referring herein to the nitrogen content of a feedstock, product or other hydrocarbon stream, the presented concentration is the value for the nitrogen content as determined by the test method ASTM D5762-12 entitled “Standard Test Method for Nitrogen in Petroleum and Petroleum Products by Boat-Inlet Chemiluminescence.” The units used in this specification, such as ppmw or wt. %, when referring to nitrogen content are the values that correspond to those as reported under ASTM D5762, i.e., in micrograms/gram (μg/g) nitrogen, but converted into referenced unit.

The additive-impregnated composition of the invention may be employed as a part of any suitable reactor system that provides for contacting it or its derivatives with the distillate feedstock under suitable hydrodesulfurization conditions that may include the presence of hydrogen and an elevated total pressure and temperature. Such suitable reaction systems can include fixed catalyst bed systems, ebullating catalyst bed systems, slurried catalyst systems, and fluidized catalyst bed systems.

The preferred reactor system is that which includes a fixed bed of the inventive catalyst contained within a reactor vessel equipped with a reactor feed inlet means, such as a feed nozzle, for introducing the distillate feedstock into the reactor vessel, and a reactor effluent outlet means, such as an effluent outlet nozzle, for withdrawing the reactor effluent or the treated hydrocarbon product or the ultra-low sulfur distillate product from the reactor vessel.

The hydrotreating process (either hydrodenitrogenation or hydrodesulfurization, or both) generally operates at a hydrotreating reaction pressure in the range of from 689.5 kPa (100 psig) to 13,789 kPa (2000 psig), preferably from 1896 kPa (275 psig) to 10,342 kPa (1500 psig), and, more preferably, from 2068.5 kPa (300 psig) to 8619 kPa (1250 psig).

The hydrotreating reaction temperature is generally in the range of from 200° C. (392° F.) to 420° C. (788° F.), preferably, from 260° C. (500° F.) to 400° C. (752° F.), and, most preferably, from 320° C. (608° F.) to 380° C. (716° F.).

The flow rate at which the distillate feedstock is charged to the reaction zone of the inventive process is generally such as to provide a liquid hourly space velocity (LHSV) in the range of from 0.01 hr⁻¹ to 10 hr⁻¹. The term “liquid hourly space velocity”, as used herein, means the numerical ratio of the rate at which the distillate feedstock is charged to the reaction zone of the inventive process in volume per hour divided by the volume of catalyst contained in the reaction zone to which the distillate feedstock is charged. The preferred LHSV is in the range of from 0.05 hr⁻¹ to 5 hr⁻¹, more preferably, from 0.1 hr⁻¹ to 3 hr⁻¹. and, most preferably, from 0.2 hr⁻¹ to 2 hr⁻¹.

It is preferred to charge hydrogen along with the distillate feedstock to the reaction zone of the inventive process. In this instance, the hydrogen is sometimes referred to as hydrogen treat gas. The hydrogen treat gas rate is the amount of hydrogen relative to the amount of distillate feedstock charged to the reaction zone and generally is in the range upwardly to 1781 m³/m³ (10,000 SCF/bbl). It is preferred for the treat gas rate to be in the range of from 89 m³/m³ (500 SCF/bbl) to 1781 m³/m³ (10,000 SCF/bbl), more preferably, from 178 m³/m³ (1,000 SCF/bbl) to 1602 m³/m³ (9,000 SCF/bbl), and, most preferably, from 356 m³/m³ (2,000 SCF/bbl) to 1425 m³/m³ (8,000 SCF/bbl).

The desulfurized distillate product yielded from the process of the invention has a low or reduced sulfur concentration relative to the distillate feedstock. A particularly advantageous aspect of the inventive process is that it is capable of providing a deeply desulfurized diesel product or an ultra-low sulfur diesel product. As already noted herein, the low sulfur distillate product can have a sulfur concentration that is less than 50 ppmw or any of the other noted sulfur concentrations as described elsewhere herein (e.g., less than 15 ppmw, or less than 10 ppmw, or less than 8 ppmw).

If the hydrotreated distillate product yielded from the process of the invention has a reduced nitrogen concentration relative to the distillate feedstock, it typically is at a concentration that is less than 50 ppmw, and, preferably, the nitrogen concentration is less than 20 ppmw or even less than 15 or 10 ppmw.

The following examples are presented to further illustrate certain aspects of the invention, but they are not to be construed as limiting the scope of the invention.

### EXAMPLE 1 (CATALYST A)

This Example 1 describes the preparation of an embodiment of the inventive polar additive-impregnated compositions made by a method using citric acid as an additional component of the metals impregnation solution.

A cobalt molybdenum (CoMo) catalyst utilizing citric acid is prepared as follows: 19.94 grams of molybdenum trioxide, 6.06 grams of cobalt hydroxide, 10.22 grams of citric acid mono hydrate, 7.91 grams of 85% phosphoric acid solution, and 45 ml of deionized water are heated with stirring to 180° F. until a clear solution results. The volume of the solution is adjusted with additional deionized water such that the incipient wetness impregnation onto the support is at a 98% pore filling factor. The solution is cooled and impregnated onto 60 grams of a gamma-alumina extrudate of 1.3 mm diameter with a tri-lobe shape. The support is then aged for 2-12 hours at ambient conditions in a sealed vessel followed by drying in air at 125° C. for 2 hours. The sample is subsequently dried in air at 160° C. for 1 hour to surpass the melting point of the citric acid ingredient. After cooling, the sample is then impregnated by incipient wetness impregnation to a pore filling factor of 95% with a polar additive to yield the finished catalyst.

### EXAMPLE 2 (CATALYST B)

This Example 2 describes the preparation of an embodiment of the inventive polar additive-impregnated composition made by a method using gluconic acid as an additional component of the metals impregnation solution.

A cobalt molybdenum (CoMo) catalyst utilizing gluconic acid is prepared as follows: 19.94 grams of molybdenum trioxide, 6.06 grams of cobalt hydroxide, 20.44 grams of 50% gluconic acid solution, 7.91 grams of 85% phosphoric acid solution, and 45 ml of deionized water are heated with stirring to 180° F. until a clear solution results. The volume of the solution is adjusted with additional deionized water such that the incipient wetness impregnation onto the support is at a 98% pore filling factor. The solution is cooled and impregnated onto 60 grams of a gamma-alumina extrudate of 1.3 mm diameter with a tri-lobe shape. The support is then aged for 2-12 hours at ambient conditions in a sealed vessel followed by drying in air at 85° C. for 3 hours. The sample is subsequently dried in air at 140° C. for 30 min to surpass the melting point of the gluconic acid ingredient. After cooling, the sample is then impregnated by incipient wetness impregnation to a pore filling factor of 95% with a polar additive to yield the finished catalyst.

### EXAMPLE 3 (REFERENCE CATALYST)

This Example 3 describes the preparation of the reference catalyst composition by a method that uses no gluconic acid or citric acid as an additional component in the metals impregnation solution.

A cobalt molybdenum (CoMo) reference catalyst is prepared as follows: 19.94 grams of molybdenum trioxide, 6.06 grams of cobalt hydroxide, 7.91 grams of 85% phosphoric acid solution, and 45 ml of deionized water are heated with stirring to 180° F. until a clear solution results. The volume of the solution is adjusted with additional deionized water such that the incipient wetness impregnation onto the support is at a 98% pore filling factor. The solution is cooled and impregnated onto 60 grams of a gamma-alumina extrudate of 1.3 mm diameter with a tri-lobe shape. The support is then aged for 2-12 hours at ambient conditions in a sealed vessel followed by drying in air at 125° C. for 2 hours. After cooling, the sample is then impregnated by incipient wetness impregnation to a pore filling factor of 95% with a polar additive to yield the finished catalyst.

### EXAMPLE 4

This Example 4 describes the procedure used to measure the performance of the catalyst compositions prepared as described in Examples 1-3 in the hydrotreating of distillate feedstock.

A tubular reactor composed of stainless steel is loaded with 50 milliliters of the catalyst to be tested diluted with 140 milliliters of 70 mesh silicon carbide.

The catalyst is sulfided using a straight-run distillate feedstock spiked to 2.5 wt % sulfur content using tert-nonylpolysulfide, dimethyldisulfide, or similar sulfiding agent. Pure hydrogen gas flow is established over the catalyst bed at 10.69 liters per hour at ambient temperature. The reactor is pressurized to 600 psig and the temperature is ramped to 300° F. at 45° F. per hour. The reactor is held at 300° F. for 3 hours. The spiked straight-run feed is then introduced to the reactor at 1.5 liquid hourly space velocity. The reactor temperature is then ramped to 600° F. at 45° F. per hour and held for 3 hours. The temperature was then lowered to 400° F. and straight-run distillate test feed was introduced at 1.0 liquid hourly space velocity continuing to maintain hydrogen flow at 10.69 liters per hour. The temperature is then ramped up to 600° F. at 20° F. per hour and the to 640° F. at 10° F. per hour. The catalyst is operated isothermally at this temperature for approximately 400 hours with daily sampling of product sulfur levels to determine catalyst activity.

### EXAMPLE 5

This Example 5 presents summary results of the performance testing of Catalyst A, Catalyst B and Reference Catalyst that provide for comparisons of the performance of these catalyst compositions.

FIG. 1 presents a plot of the adjusted WABT over time provided by Catalyst A and the Reference Catalyst that is required to yield a desulfurized gas oil product having 10 ppm concentration of sulfur. As may be observed from the two plots, the adjusted WABT for Catalyst A is 6 to 7° F. lower than that of the Reference Catalyst. This demonstrates that Catalyst A exhibits a significantly greater activity for hydrodesulfurization than that of the Reference Catalyst.

FIG. 2 presents similar results to those presented in FIG. 1. Shown is a plot of the adjusted WABT over time provided by Catalyst B and the Reference Catalyst that is required to yield a desulfurized gas oil product having 10 ppm concentration of sulfur. As may be observed from the two plots, the adjusted WABT for Catalyst B is as much as 5° F. lower than that of the Reference Catalyst. This demonstrates that Catalyst B exhibits a significantly greater activity for hydrodesulfurization than that of the Reference Catalyst.

These results show that the catalytic activity of a polar additive-containing hydroprocessing catalyst is significantly enhanced by use of a molten organic acid as an additional component of the metals impregnation solution used in preparing the catalyst.

That which is claimed is:

1. A method of making a hydroprocessing catalyst composition, wherein said method comprises:
incorporating an impregnation solution, comprising a Group 9/10 metal component, a Group 6 metal component, and an organic acid compound, into a support material to provide an impregnated support material;
drying said impregnated support material at a drying temperature that is in the range of from above the melting temperature of said organic acid compound and below the boiling temperature of said organic acid compound to provide a dried impregnated support material; and
incorporating a polar additive having a dipole moment of at least 0.45 into said dried impregnated support material to thereby provide an additive-impregnated composition.

2. A method as recited in claim 1, wherein said organic acid compound is selected from the group of carboxylic compounds consisting of di or tri carboxylic acid compounds having at least 3 up to 10 carbon atoms and which may or may not be substituted with one or more hydroxyl groups, wherein the molar mass of the carboxylic acid compound is in the range of from 100 to 250 g/mol, the melting point of the carboxylic acid compound is in the range of from 100 to 200° C., and the carboxylic acid compound is soluble in water.

3. A method as recited in claim 2, wherein said organic acid compound is either gluconic acid or citric acid.

4. A method as recited in claim 3, wherein said impregnation solution is an aqueous solution including a metal salt of said Group 9/10 metal component that is selected from the group consisting of cobalt and nickel, wherein said Group 9/10 metal component is present in said aqueous solution in an amount in the range of from 0.5 wt. % to 20 wt. % of said aqueous solution, a metal salt of said Group 6 metal component selected from the group consisting of molybdenum and tungsten, wherein said Group 6 metal component is present in said aqueous solution in an amount in the range of from 5 wt. % to 50 wt. % of said aqueous solution, and said organic acid compound present in said aqueous solution in an amount upwardly to 50 wt. % of said aqueous solution.

5. A method as recited in claim 1, wherein said polar additive has a boiling point in the range of from 50° C. to 275° C.

6. A method as recited in claim 5, wherein said polar additive is selected from a heterocompound group consisting of heterocompounds.

7. A method as recited in claim 6, wherein said heterocompound group excludes sulfur-containing compounds.

8. A method as recited in claim 7, wherein said heterocompound group excludes paraffin and olefin hydrocarbon compounds.

9. A method as recited in claim 9, wherein said polar additive is a heterocyclic compound of the formula CₓHₙN_{y}O_{z}, wherein x is an integer of 3 or larger, y is either zero or an integer in the range of from 1 to 3, z is either zero or an integer in the range of from 1 to 3, and n is the number of hydrogen atoms required to fill the remaining bonds with carbon atoms of the molecule.

10. A method as recited in claim 10, wherein said heterocyclic compound is one containing either nitrogen or oxygen as the heteroatom member of its ring providing either a lactam structure or a cyclic ester structure.

11. A method as recited in claim 11, wherein said polar additive is either propylene carbonate or N-methylpyrrolidone, or a combination of both.

12. A method as recited in claim 1, further comprising: contacting said additive-impregnated composition under suitable hydrogen treatment conditions with hydrogen to thereby provide a hydrogen-treated composition.

13. A method as recited in claim 4, further comprising: contacting said additive-impregnated composition under suitable hydrogen treatment conditions with hydrogen to thereby provide a hydrogen-treated composition.

14. A method as recited in claim 11, further comprising: contacting said additive-impregnated composition under suitable hydrogen treatment conditions with hydrogen to thereby provide a hydrogen-treated composition.

15. A hydroprocessing catalyst composition, comprising: a dried, impregnated support material, wherein said impregnated support material comprises a support material, a Group 9/10 metal component, a Group 6 metal component, and an organic acid compound, wherein said impregnated support material has been dried at a drying temperature in the range of from above the melting temperature of said organic acid compound and below the boiling temperature of said organic acid compound to provide said dried, impregnated support material; and a polar additive having a dipole moment of at least 0.45 incorporated into said dried, impregnated support material.

16. A composition as recited in 15, wherein said composition, comprising said dried, impregnated support material and said polar additive, is further treated with hydrogen.

17. A composition as recited in claim 15, wherein said polar additive has a boiling point in the range of from 50° C. to 275° C.

18. A composition as recited in claim 15, wherein said polar additive is selected from a heterocompound group consisting of heterocompounds.

19. A composition as recited in claim 15, wherein said heterocompound group excludes sulfur-containing compounds.

20. A composition as recited in claim 15, wherein said heterocompound group excludes paraffin and olefin hydrocarbon compounds.

21. A composition as recited in claim 15, wherein said polar additive is a heterocyclic compound of the formula CₓHₙN_{y}O_{z}, wherein x is an integer of 3 or larger, y is either zero or an integer in the range of from 1 to 3, z is either zero or an integer in the range of from 1 to 3, and n is the number of hydrogen atoms required to fill the remaining bonds with carbon atoms of the molecule.

22. A composition as recited in claim 21, wherein said heterocyclic compound is one containing either nitrogen or oxygen as the heteroatom member of its ring providing either a lactam structure or a cyclic ester structure.

23. A composition as recited in claim 15, wherein said polar additive is either propylene carbonate or N-methylpyrrolidone, or a combination of both.

24. A composition as recited in claim 15, wherein said organic acid compound is selected from the group of carboxylic compounds consisting of di or tri carboxylic acid compounds having at least 3 up to 10 carbon atoms and which may or may not be substituted with one or more hydroxyl groups, wherein the molar mass of the carboxylic acid compound is in the range of from 100 to 250 g/mol, the melting point of the carboxylic acid compound is in the range of from 100 to 200° C., and the carboxylic acid compound is soluble in water.

25. A composition as recited in claim 15, wherein said organic acid compound is either gluconic acid or citric acid.

26. A composition as recited in claim 15, wherein said Group 9/10 metal component is selected from the group consisting of cobalt and nickel, and wherein said Group 9 and Group 10 metal component is present in said composition in an amount in the range of from 0.5 wt. % to 20 wt. %.

27. A composition as recited in claim 15, wherein said Group 6 metal component selected from the group consisting of molybdenum and tungsten, and wherein said Group 6 metal component is present in said composition in an amount in the range of from 5 wt. % to 50 wt. %.

28. A composition made by the method of claim 1.

29. A process, comprising: contacting under hydroprocessing process conditions a hydrocarbon feedstock with the composition of claim 15.
